(19) United States
(12) Patent Application Publication
(10) Pub. No.: US 2024/0057882 A1
(43) Pub. Date: Feb. 22, 2024

(54) TORSO SENSOR DEVICE

(71) Applicant: BIOINTELLISENSE, INC., Golden, CO (US)

(72) Inventors: Harley Gordon White, Fort Collins, CO (US); David Jonq Wang, Palo Alto, CA (US)

(21) Appl. No.: 18/452,454

(22) Filed: Aug. 18, 2023

Related U.S. Application Data
(60) Provisional application No. 63/371,830, filed on Aug. 18, 2022.

(51) Int. Cl. A61B 5/024; A61B 5/021; A61B 5/349; A61B 5/00

(52) U.S. Cl. CPC A61B 5/02433; A61B 5/02116; A61B 5/349; A61B 5/6823

(57) ABSTRACT

In an example, a torso sensor device includes a housing, first and second optical sensors, and a processor. The housing is configured to be coupled to a subject's torso. The first optical sensor is disposed in the housing. The second optical sensor is disposed in the housing spaced apart from the first optical sensor. The processor is electrically coupled to each of the first and second optical sensors and is configured to control the first and second optical sensors to generate oxygen saturation measurements of the subject.

### CROSS-REFERENCE TO RELATED APPLICATION

This application claims the benefit of and priority to U.S. Provisional Patent App. No. 63/731,830 filed Aug. 18, 2022. The 63/731,830 application is incorporated herein by reference in its entirety.

### FIELD

The embodiments discussed herein are related to a torso sensor device.

### BACKGROUND

Unless otherwise indicated herein, the materials described herein are not prior art to the claims in the present application and are not admitted to be prior art by inclusion in this section.

Pulse oximeters use light emitting diodes (LEDs) of different wavelengths together with a light-sensitive sensor to measure the absorption of red and infrared light at a measurement site of a subject. Since oxygenated hemoglobin absorbs more infrared (IR) light than red light and deoxygenated hemoglobin absorbs more red light than IR light, peripheral oxygen saturation (SpO₂) of the subject's blood may be determined by measuring the absorbance of red and IR light and calculating SpO₂ based on the absorbance of the two wavelengths of light. Alternatively, SpO₂ may be calculated from reflectance of red light and IR light at least in the case of a reflection-based pulse oximeter. Pulse oximeters may also be referred to as SpO₂ sensors.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, a torso sensor device includes a housing, first and second optical sensors, and a processor. The housing is configured to be coupled to a subject's torso. The first optical sensor is disposed in the housing. The second optical sensor is disposed in the housing spaced apart from the first optical sensor. The processor is electrically coupled to each of the first and second optical sensors and is configured to control the first and second optical sensors to generate oxygen saturation measurements of the subject.

In another example embodiment, a method to measure oxygen saturation includes generating a first signal of a subject using a first optical sensor of a torso sensor device. The method includes generating a second signal of the subject using a second optical sensor of the torso sensor device. The second optical sensor is spaced apart from the first optical sensor such that the first and second signals are generated from different locations of the subject. The method includes determining which of the first or second signals is better. The method includes generating an oxygen saturation measurement of the subject based on the better signal.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an example operating environment for a torso sensor device;

FIGS. 2A and 2B depict two different placements of a torso sensor device relative to a human rib cage;

FIGS. 3A and 3B depict two different placements of a torso sensor device relative to the rib cage of FIGS. 2A and 2B where the torso sensor device has two optical sensors;

FIG. 4 depicts placement of the torso sensor device of FIGS. 3A and 3B with sensor axes aligned generally parallel to the second and third ribs and/or the second intercostal space (ICS) of the rib cage;

FIGS. 5A-5D illustrate an example torso sensor device that may be implemented in the environment of FIG. 1;

FIG. 6 illustrates another example torso sensor device that may be implemented in the environment of FIG. 1;

FIG. 7 is a top front perspective view of an example optical sensor that may be implemented in any of the torso sensor devices herein;

FIG. 8 illustrates various rib models and an example configuration of a torso sensor device;

FIG. 9A is a flowchart of a method to measure oxygen saturation;

FIG. 9B illustrates example photoplethysmographic traces from two different optical sensors of the same torso sensor device; and

FIG. 10 is a block diagram illustrating an example computing device, all arranged in accordance with at least one embodiment described herein.

### DETAILED DESCRIPTION OF SOME EXAMPLE EMBODIMENTS

Many SpO₂ sensors have an array of light emitting diodes (LEDs). These SpO₂ sensors are typically large and not immune to motion. Other SpO₂ sensors may be smaller and/or less affected by motion but may be more sensitive to placement on a subject. For instance, if the SpO₂ sensor is placed over an area of the subject with limited blood flow (such as directly over a rib), blood flow at the measurement site may be insufficient to generate accurate SpO₂ measurements.

To mitigate placement sensitivity of such SpO₂ sensors, some embodiments herein incorporate two (or more) SpO₂ sensors into a single torso sensor device with the SpO₂ sensors spaced apart from each other. The spacing of the SpO₂ sensors may be configured to improve a likelihood of at least one of the SpO₂ sensors being positioned over a measurement site of the subject that has sufficient blood flow (such as directly over an ICS between two ribs) for accurate SpO₂ measurements. For example, the center-to-center spacing of the SpO₂ sensors may be approximately half of a rib-to-rib spacing (center of rib to center of adjacent rib) of the subject so that if one of the SpO₂ sensors is directly over one of the ribs, the other SpO₂ sensor is directly over an adjacent ICS. The optical sensors are positioned at a first surface of the torso sensor device to be proximate to the subject's skin when the torso sensor device is coupled to the subject. The torso sensor device includes an opposing second surface that is visible when the torso sensor device is coupled to the subject. The second surface of the torso sensor device may include one or more alignment guides to use as a visual aid in positioning and/or aligning one or more of the SpO₂ sensors between ribs to be directly over a corresponding ICS.

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

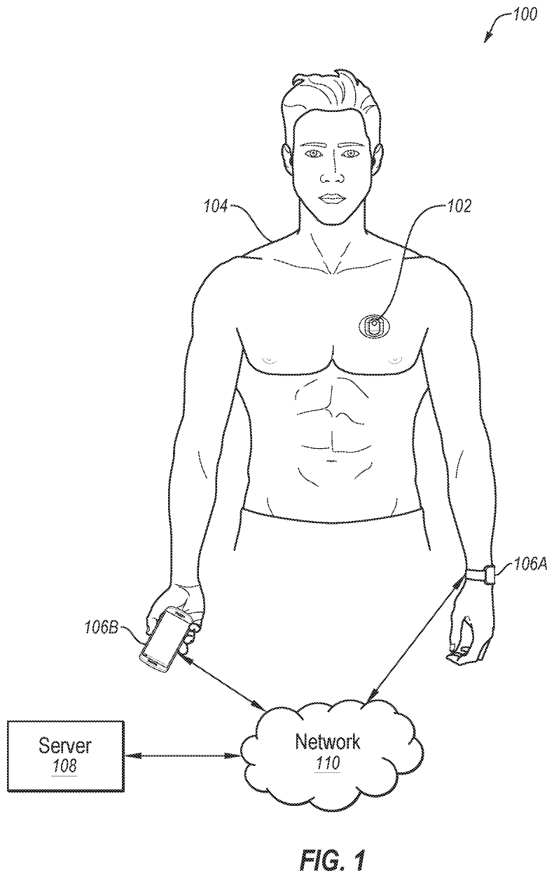

FIG. 1 illustrates an example operating environment 100 (hereinafter “environment 100”) for a torso sensor device 102, arranged in accordance with at least one embodiment described herein. The environment 100 includes a subject 104 and one or more personal electronic devices 106A, 106B (hereinafter collectively “personal electronic devices 106” or generically “personal electronic device 106”). The environment 100 may additionally include a server 108 and a network 110.

In general, the torso sensor device 102 may be attached to the subject 104, such as to the torso of the subject 104, to detect oxygen saturation of hemoglobin or other measurement parameter. In some embodiments, attaching the torso sensor device 102 to the torso of the subject 104 may include attaching the torso sensor device 102 somewhere on the chest wall of the subject, such as on the right anterior chest wall, the left anterior chest wall, the right posterior chest wall, and/or the left posterior chest wall. In an example, the torso sensor device 102 is configured specifically to detect SpO₂ of the subject 104. In some embodiments, the quality of measurements generated by the torso sensor device 102 may depend on the position of the torso sensor device 102 relative to ribs and/or ICS s of the rib cage of the subject 104, as discussed in more detail elsewhere herein.

The personal electronic devices 106 may each include a desktop computer, a laptop computer, a tablet computer, a smartphone, a wearable electronic device (e.g., smart watch, activity tracker, headphones, ear buds, etc.), or other personal electronic device. In the illustrated example, the personal electronic device 104A is a smart watch and the personal electronic device 104B is a smartphone. In some embodiments, the personal electronic devices 104 may collect measurement data from the torso sensor device 102 for use and/or analysis on the personal electronic devices 104.

Alternatively or additionally, the measurement data generated by the torso sensor device 102 and/or data derived therefrom may be uploaded, e.g., periodically, by torso sensor device 102 to the server 108. In some embodiments, one or more of the personal electronic devices 106 or another device may act as a hub that collects measurement data or data derived therefrom from the torso sensor device 102 and/or other personal electronic devices 106 and uploads the measurement data or data derived therefrom to the server 108. For example, the hub may collect data over a local communication scheme (WI-FI, BLUETOOTH, near-field communications (NFC), etc.) and may transmit the data to the server 108. In some embodiments, the hub may act to collect the data and periodically provide the data to the server 108, such as once per week. An example hub and associated methods and devices are disclosed in U.S. Pat. No. 10,743,091, which is incorporated herein by reference.

The server 108 may include a collection of computing resources available in the cloud and/or a discrete server computer. The server 108 may be configured to receive measurement data and/or data derived from measurement data from one or more of the personal electronic devices 106 and/or from the torso sensor device 102. Alternatively or additionally, the server 108 may be configured to receive from the torso sensor device 102 (e.g., directly or indirectly via a hub device) relatively small portions of the measurement data, or even larger portions or all of the measurement data. The server 108 may use and/or analyze the data, e.g., to detect and/or monitor oxygen saturation of the subject 104 or other biological parameters (e.g., heart rate, arrhythmia, or the like). Alternatively or additionally, the server 108 may store the measurement data in an account of the subject 104 and make the measurement data or data derived therefrom available to the subject 104, a healthcare provider, or other individuals, e.g., as authorized by the subject 104 e.g., via an online portal.

The network 110 may include one or more wide area networks (WANs) and/or local area networks (LANs) that enable the personal electronic devices 106, the server 108, and/or the torso sensor device 102 to communicate with each other. In some embodiments, the network 110 includes the Internet, including a global internetwork formed by logical and physical connections between multiple WANs and/or LANs. Alternately or additionally, the network 110 may include one or more cellular radio frequency (RF) networks and/or one or more wired and/or wireless networks such as 801.xx networks, BLUETOOTH access points, wireless access points, IP-based networks, or other suitable networks. The network 110 may also include servers that enable one type of network to interface with another type of network.

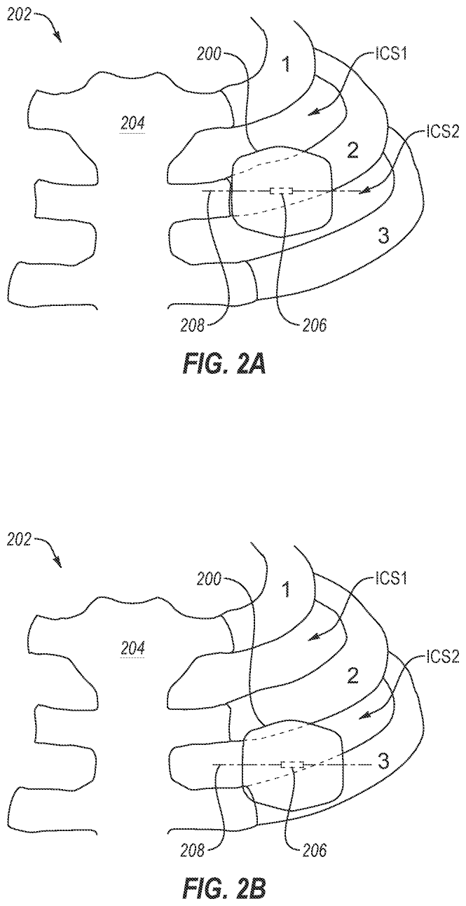

FIGS. 2A and 2B depict two different placements of a torso sensor device 200 relative to a human rib cage 202 (hereinafter “rib cage 202”), arranged in accordance with at least one embodiment described herein. The torso sensor device 200 may include, be included in, or correspond to the torso sensor device 102 of FIG. 1. In practice, there is generally intervening skin, muscle, arteries, veins, or the like between the torso sensor device 200 and the rib cage 202 which have been omitted from FIGS. 2A and 2B for illustration purposes.

As illustrated, the rib cage 202 includes a sternum 204 and various ribs extending therefrom, only three of which are illustrated in FIGS. 2A and 2B for simplicity. In FIGS. 2A, and 2B, short-dashed lines represent approximate boundaries of portions of ribs that are not visible through the torso sensor device 200. FIGS. 2A and 2B depict the first rib (“1” in FIGS. 2A and 2B), the second rib (“2” in FIGS. 2A and 2B), and the third rib (“3” in FIGS. 2A and 2B) on the anatomical left side of the rib cage 202. The first and second ribs are separated by the first ICS (“ICS1” in FIGS. 2A and 2B) while the second and third ribs are separated by the second ICS (“ICS2” in FIGS. 2A and 2B). While FIGS. 2A and 2B illustrate only the top three ribs and top two ICSs on the anatomical left side of the rib cage 202, it is understood that the rib cage 202 typically includes additional ribs and ICSs beyond those illustrated (e.g., generally 12 pairs of ribs and 11 ICSs in humans).

The torso sensor device 200 includes a first surface, sometimes referred to as a bottom surface, that may be positioned adjacent to the torso of the subject 104 (FIG. 1) when coupled to the subject 104. The torso sensor device 200 also includes a second surface, sometimes referred to as a top surface, opposite the first surface and which may be visible when the torso sensor device 200 is coupled to the subject 104. The torso sensor device 200 further includes at least one optical sensor positioned at the first surface of the torso sensor device 200 at a location of the torso sensor device 200 designated at 206. Insofar as the first surface of the torso sensor device 200 is not visible when coupled to the subject 104, the location 206 is more particularly an approximate footprint or outline of the optical sensor as projected from the first surface to the second surface of the torso sensor device 200. In the discussion that follows, the optical sensor positioned at the location 206 of the torso sensor device 200 is referred to as the “optical sensor 206”. Each optical sensor of the torso sensor device 200 may generally include one or more optical emitters and one or more optical detectors. In some embodiments, a line between one of the optical emitters and one of the optical detectors defines a sensor axis 208 of the optical sensor 206, the sensor axis 208 being depicted with a long-dash line in FIGS. 2A and 2B. Alternatively or additionally, the line that defines the sensor axis 208 may be between a first point located at a center or centroid of two or more optical emitters and a second point located at a center or centroid of two or more optical detectors. Alternatively or additionally, the line that defines the sensor axis 208 of the optical sensor 206 may be determined in some other manner.

In FIG. 2A, the placement of the torso sensor device 200 relative to the rib cage 202 positions the optical sensor 206 over the second rib of the rib cage 202. In actual use, the amount of blood flow past the optical sensor 206 in this position may be limited due to the placement of the torso sensor device 200 over the second rib or any other rib. As a result of the relatively limited blood flow over the second rib (and other ribs) that moves past the optical sensor 206 when the torso sensor device 200 is positioned as shown in FIG. 2A (or with the optical sensor 206 over another rib), oxygen saturation measurements generated with this placement may be poor.

In FIG. 2B, the placement of the torso sensor device 200 relative to the rib cage 202 positions the optical sensor 206 over the second ICS of the rib cage 202. The ICS s of the rib cage 202, including the second ICS, contain intercostal muscles and neurovascular bundles containing nerves, arteries, and veins. As such, blood flow that moves past the optical sensor 206 when positioned over the second ICS (or other ICS) as in FIG. 2B may be greater than when the optical sensor 206 is positioned over a rib as in FIG. 2A. The greater blood flow when the optical sensor 206 is positioned over the second ICS (or other ICS) may result in improved oxygen saturation measurements compared to when the optical sensor 206 is positioned over a rib.

Moreover, the placement of the torso sensor device 200 relative to the rib cage 202 may change over time after the torso sensor device 200 has been coupled to the subject 104. For example, if the subject 104 raises their left arm up or makes other movements, the torso sensor device 200 may move relative to the rib cage, such as from the placement of FIG. 2B to the placement of FIG. 2A. As a result, the quality of oxygen saturation measurements generated by the torso sensor device 200 may vary depending on movement of the user that causes the optical sensor 206 to move relative to the rib cage 202.

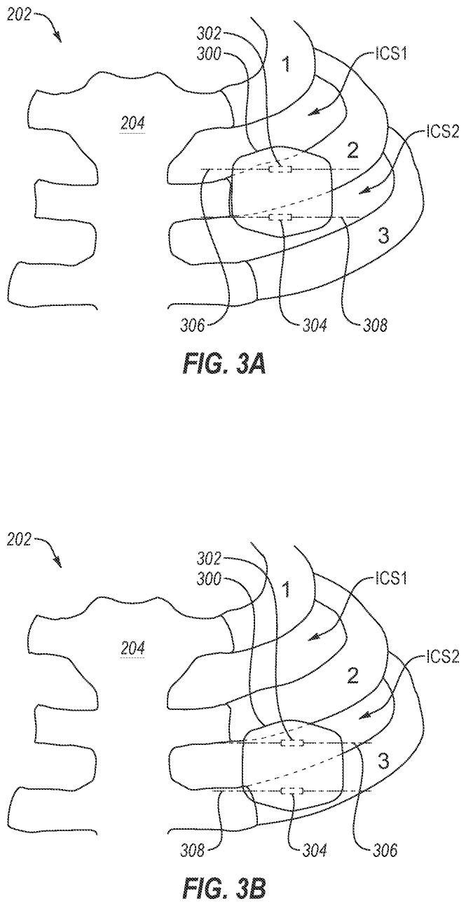

To improve the likelihood of generating oxygen saturation measurements using an optical sensor positioned over an ICS of the subject 104, torso sensor devices herein may include two (or more) optical sensors that are spaced apart from each other. The spacing may be selected to reduce or eliminate the likelihood of both of the optical sensors being simultaneously positioned over a rib and/or such that if one of the optical sensors is over a rib, the other is likely to be over an ICS. In these and other embodiments, the torso sensor device may include one or more alignment guides that identify the location and/or alignment of the sensor axis of each optical sensor as a visual aid for the subject 104, a healthcare provider, or other person to align at least one of the sensor axes to an ICS of the subject 104. This may improve the likelihood of the torso sensor device being placed on the torso of the subject with at least one of the optical sensors over an ICS of the subject. Alternatively or additionally, the torso sensor device may analyze signals generated by each of the optical sensors when generating oxygen saturation measurements and may use the signal with the largest AC component or other parameter to generate the oxygen saturation measurements. Accordingly, when movement or other action of the subject 104 results in one of the optical sensors moving from over an ICS to over a rib, this same movement or other action may result in another of the optical sensors moving from over a rib to over an ICS and its signal may then be used to generate the oxygen saturation measurements. An example of a torso sensor device with two optical sensors is depicted in FIGS. 3A and 3B.

FIGS. 3A and 3B depict two different placements of a torso sensor device 300 relative to the rib cage 202 where the torso sensor device 300 has two optical sensors, arranged in accordance with at least one embodiment described herein. The torso sensor device 300 may include, be included in, or correspond to the torso sensor device 102 of FIG. 1. Similar to FIGS. 2A and 2B, in FIGS. 3A and 3B the intervening skin, muscle, arteries, veins, or the like between the torso sensor device 300 and the rib cage 202 have been omitted for illustration purposes. Moreover, short-dashed lines in FIGS. 3A and 3B represent approximate boundaries of portions of ribs that are not visible through the torso sensor device 300.

In the example of FIGS. 3A and 3B, the torso sensor device 300 includes two optical sensors positioned at the first surface of the torso sensor device 300 at locations of the torso sensor device 300 designated at 302 and 304. Insofar as the first surface of the torso sensor device 300 is not visible when coupled to the subject 104, the locations 302 and 304 are more particularly approximate footprints or outlines of the optical sensors as projected from the first surface to the second surface of the torso sensor device 300. In the discussion that follows, the optical sensor positioned at the location 302 of the torso sensor device 300 is referred to as the “optical sensor 302” and the optical sensor positioned at the location 304 of the torso sensor device 300 is referred to as the “optical sensor 304”. Each of the optical sensors 302, 304 may generally include one or more optical emitters and one or more optical detectors. Each of the optical sensors 302, 304 includes a sensor axis 306, 308 defined with respect to the one or more optical emitters and the one or more optical detectors as described above.

In FIG. 3A, the placement of the torso sensor device 300 relative to the rib cage 202 positions the optical sensor 302 over the second rib and the optical sensor 304 over the second ICS. Thus, while there may be relatively limited blood flow past the optical sensor 302 positioned over the second rib, there may be greater blood flow past the optical sensor 304 positioned above the second ICS. In this example, good oxygen saturation measurements may be generated based on the signal from the optical sensor 304 even though oxygen saturation measurements generated based on the signal from the optical sensor 302 may be poor.

In FIG. 3B, the placement of the torso sensor device 300 relative to the rib cage 202 positions the optical sensor 302 over the second ICS and the optical sensor 304 over the third rib. Similar to the placement of FIG. 3A, the placement of FIG. 3B allows good oxygen saturation measurements to be generated based on the signal from the optical sensor over the ICS (the optical sensor 302 in FIG. 3B) even though oxygen saturation measurements generated based on the signal from the optical sensor over the rib (the optical sensor 304 in FIG. 3B) may be poor.

The inclusion of the two optical sensors 302, 304 in the torso sensor device 300 allows good oxygen saturation measurements to be obtained even though the placement of the torso sensor device 300 relative to the rib cage 202 may change over time. For example, if the subject raises their left arm up or makes other movements that result in movement of the torso sensor device from the relative placement of FIG. 3B to the relative placement of FIG. 3A, good oxygen saturation measurements may be obtained in both positions since one of the optical sensors 102B, 102C is over an ICS of the rib cage 202 in each position.

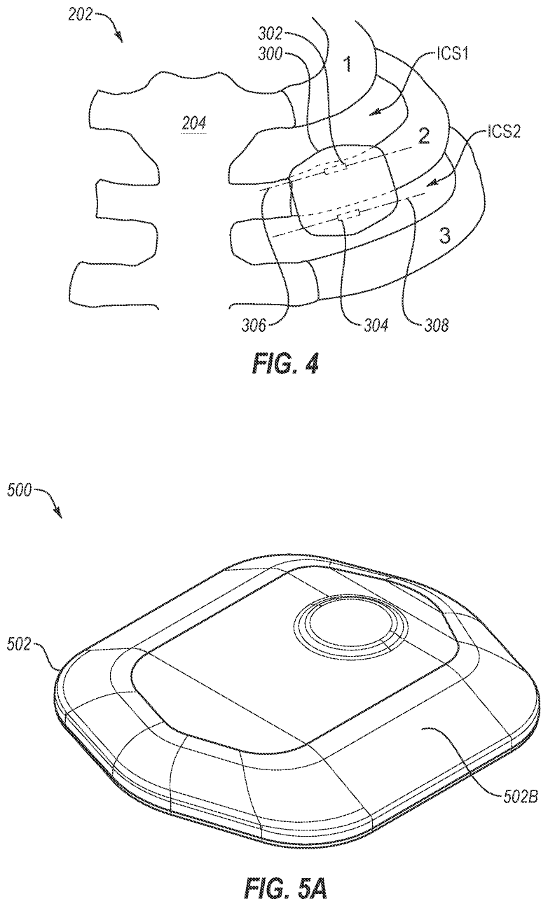

In the examples of FIGS. 2A-3B, the optical sensors 206, 302, 304 are generally elongate and the one or more optical emitters may be positioned on an opposite side of the generally elongate optical sensor 206, 302, 304 from the one or more optical detectors. Positioning either end of the generally elongate optical sensors 206, 302, 304 such that the one or more optical emitters (or the one or more optical detectors) is positioned over a rib while the one or more optical detectors (or the one or more optical emitters) is positioned over an ICS may degrade measurements. Accordingly, in some embodiments the torso sensor device 300 may be angled or rotated relative to the rib cage, as illustrated in FIG. 4. In particular, FIG. 4 depicts placement of the torso sensor device 300 with the sensor axes 302, 304 aligned generally parallel to the second and third ribs and/or the second ICS, arranged in accordance with at least one embodiment described herein. As used herein, aligned “generally parallel” refers to alignment that is exactly parallel plus or minus 25 degrees. The reference frame for “generally parallel” with respect to a rib or ICS may be a reference plane that bisects the rib or ICS into substantially equal top and bottom portions. That is, the sensor axes 302, 304 may be aligned generally parallel to the second and third ribs or the ICS if aligned exactly parallel plus or minus 25 degrees relative to the reference plane. Placement of the torso sensor device 300 relative to the rib cage 202 with the sensor axes aligned generally parallel to the rib(s) and/or ICS(s) nearest to the torso sensor device 300 may reduce the likelihood of either of the optical sensors 302, 304 being partially over a rib and partially over an ICS.

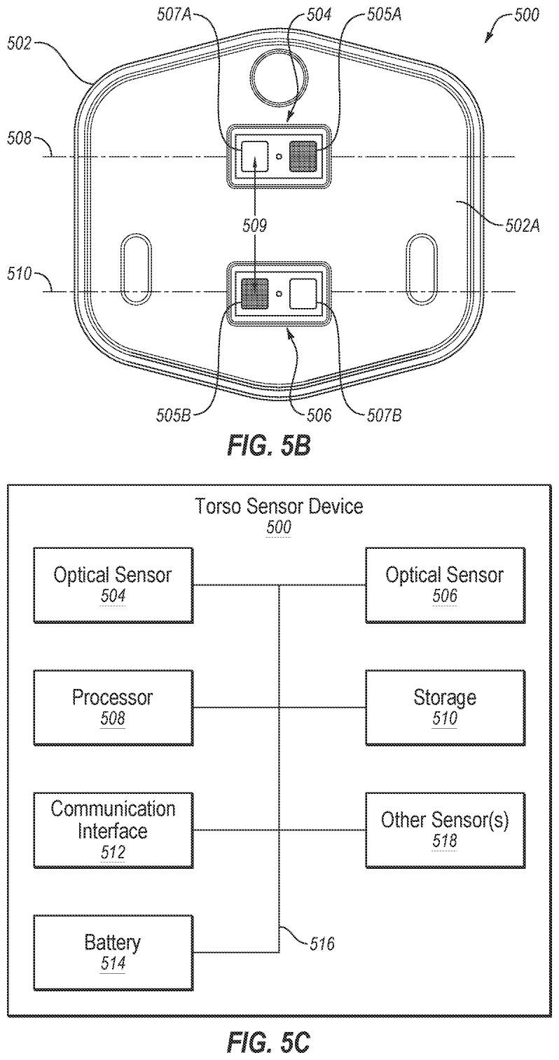

FIGS. 5A-5D illustrate an example torso sensor device 500 that may be implemented in the environment 100 of FIG. 1, arranged in accordance with at least one embodiment described herein. FIG. 5A includes a top front perspective view of the torso sensor device 500, FIG. 5B includes a bottom view of the torso sensor device 500, FIG. 5C includes a block diagram of the torso sensor device 500, and FIG. 5D illustrates a functional diagram of the torso sensor device 500. The torso sensor device 500 may include, be included in, or correspond to the torso sensor device 102 of FIG. 1 and/or other torso sensor devices described herein.

In general, the torso sensor device 500 may include a housing 502 (FIGS. 5A-5B) and two optical sensors 504, 506 (FIGS. 5B and 5C) disposed in the housing 502. The housing 502 may include a first surface 502A (FIG. 5B) configured to be positioned against the subject's torso in use and an opposing second surface 502B (FIG. 5A). Each of the optical sensors 504, 506 may be configured to detect oxygen saturation of blood of a subject, such as of the subject 104. Each of the optical sensors 504, 506 has a corresponding sensor axis 508, 510.

Referring to FIG. 5B, the optical sensor 504 may include one or more optical emitters 505A and one or more optical detectors 507A while the optical sensor 506 may include one or more optical emitters 505B and one or more optical detectors 507B. The optical emitters 505A, 505B (hereinafter collectively “optical emitters 505”) may be monochromatic light sources, non-monochromatic or polychromatic or broadband light sources, adjustable wavelength light sources, or the like. For example, the optical emitters 505 may each include a single monochromatic light source such as a red or infrared (IR) light emitting diode (LED), multiple monochromatic light sources of different wavelengths such as a red LED, an IR LED, and/or a white LED, an adjustable wavelength light source such as a single LED adjustable between red and IR wavelengths, or any combination thereof. The optical detectors 507A, 507B (hereinafter collectively “optical detectors 507”) may each include a single optical detector, multiple optical detectors, and/or multiple optical detectors with different spectral response curves. Alternatively or additionally, each of the optical detectors 507 may include a dual photodiode, a dual junction wavelength detector, an epitaxial spectral sensor, or other suitable optical detector. In some embodiments, each of the optical detectors 507 includes a first detector and a second detector having different spectral response curves. Alternative and/or additional details regarding example optical sensors according to some embodiments herein are disclosed in U.S. Pat. No. 10,485,463 which is incorporated herein by reference.

As further illustrated in FIG. 5B, each of the sensor axes 508, 510 is defined by a reference line (not shown in FIG. 5B) from the optical emitter 505 to the optical detector 507 (or vice versa). That is, the sensor axis 508 is parallel to and includes a reference line that passes through centers of the optical emitter 505A and the optical detector 507A. Similarly, the sensor axis 510 is parallel to and includes a reference line that passes through centers of the optical emitter 505B and the optical detector 507B.

FIG. 5B additionally illustrates a center-to-center spacing 509 (hereinafter “spacing 509”) of the optical sensors 504, 506. The spacing 509 may be in a range from 5 to 150 millimeters (mm), such as 10 mm to 20 mm, 14 mm to 17 mm, 15 mm, or some other range or value in the broader range of 5 mm to 150 mm. Alternatively or additionally, the spacing 509 may be greater than a width of a typical or large male subject's second rib or third rib in the parasternal area and less than a width of a typical female or small female subject's second ICS in the parasternal area. Additional details regarding the spacing 509 are described elsewhere herein.

In some embodiments, and referring to FIG. 5C, the torso sensor device 500 may further include one or more of a processor 508, storage 510, a communication interface 512, a battery 514, a communication bus 516, one or more other sensors 518, and/or other components.

The processor 508 may include any device or component configured to monitor and/or control operation of the torso sensor device 500. In some embodiments, the processor 508 is electrically coupled to the optical sensors 504, 506 and is configured to control the first and second optical sensors to generate oxygen saturation measurements. In these and other embodiments, the processor 508 may retrieve instructions from the storage 510 and execute those instructions. As another example, the processor 508 may read the signals and/or measurement data generated by sensors (e.g., the optical sensor 504, the optical sensor 506, and/or other sensor(s) 510), may process the signals and/or measurement data to generate other measurement data, and may store any of the foregoing in the storage 510 or instruct the communication interface 512 to send any of the foregoing to another electronic device, such as the server 108 of FIG. 1. In some embodiments, the processor 508 may include an arithmetic logic unit, a microprocessor, a general-purpose controller, or some other processor or array of processors, to perform or control performance of operations as described herein. The processor 508 may be configured to process data signals and may include various computing architectures including a complex instruction set computer (CISC) architecture, a reduced instruction set computer (RISC) architecture, or an architecture implementing a combination of instruction sets. Although illustrated as a single processor 508, multiple processor devices may be included and other processors and physical configurations may be possible. The processor 508 may be configured to process any suitable number format including, but not limited to two's compliment numbers, integers, fixed binary point numbers, and/or floating point numbers, etc. all of which may be signed or unsigned. In some embodiments, the processor 508 may perform processing on the readings from the sensors prior to storing and/or communicating the readings. For example, raw analog data signals generated by the optical sensor 504, the optical sensor 506, or the other sensor(s) 518 may be down-sampled, may be converted to digital data signals, and/or may be processed in some other manner.

The storage 510 may include non-transitory computer-readable storage media or one or more non-transitory computer-readable storage mediums for carrying or having computer-executable instructions or data structures stored thereon. Such non-transitory computer-readable storage media may be any available non-transitory media that may be accessed by a general-purpose or special-purpose computer, such as the processor 508. By way of example such non-transitory computer-readable storage media may include Random Access Memory (RAM), Read-Only Memory (ROM), Electrically Erasable Programmable Read-Only Memory (EEPROM), flash memory devices (e.g., solid state memory devices), or any other non-transitory storage medium which may be used to carry or store desired program code in the form of computer-executable instructions or data structures and which may be accessed by a general-purpose or special-purpose computer. In some embodiments, the storage 510 may alternatively or additionally include volatile memory, such as a dynamic random access memory (DRAM) device, a static random access memory (SRAM) device, or the like. Combinations of the above may also be included within the scope of non-transitory computer-readable storage media. Computer-executable instructions may include, for example, instructions and data that when executed by the processor 508 cause the processor 508 to perform or control performance of a certain operation or group of operations. In some embodiments, the storage 510 may store the data signals, e.g., measurement data, generated by the optical sensor 504, the optical sensor 506, the other sensor(s) 518, and/or data derived therefrom.

The communication interface 512 may include any device or component that facilitates communication with a remote device, such as any of the personal electronic devices 106 of the subject 104, the server 108, or any other electronic device. For example, the communication interface 512 may include an RF antenna, an infrared (IR) receiver, a WI-FI chip, a BLUETOOTH chip, a cellular chip, a near-field communication (NFC) chip, or any other communication interface.

The battery 514 may include any device or component configured to provide power to the torso sensor device 500 and/or the components thereof. For example, the battery 514 may include a rechargeable battery, a disposable battery, etc. In some embodiments, the torso sensor device 500 may include circuitry, electrical wires, etc. to provide power from the battery 514 to the other components of the torso sensor device 500. In some embodiments, the battery 514 may include sufficient capacity such that the torso sensor device 500 may operate for days, weeks, or months without having the battery 514 changed or recharged. For example, the torso sensor device 500 may be configured to operate for at least two months without having the battery 514 charged or replaced.

The communication bus 516 may include any connections, lines, wires, or other components facilitating communication between the various components of the torso sensor device 500. The communication bus 516 may include one or more hardware components and may communicate using one or more protocols. Additionally or alternatively, the communication bus 516 may include wire connections between the components. In some embodiments, the torso sensor device 500 may operate in a similar or comparable manner to the embodiments described in U.S. application Ser. No. 17/485,315 filed on Sep. 24, 2021, U.S. application Ser. No. 17/349,166 filed on Jun. 16, 2021, and/or U.S. Pat. No. 11,172,909 issued Nov. 16, 2021, each of which is hereby incorporated by reference.

The one or more other sensor(s) 518 may include an electrocardiogram (ECG) sensor, a temperature sensor, a respiratory sensor, an accelerometer, a microphone, a gyrometer sensor, a blood pressure sensor, an optical spectrometer sensor, an electro-chemical sensor, an oxygen saturation sensor, an electrodermal activity (EDA) sensor, a volatile organic compound (VOC) sensor, a spectrometer, or any combination thereof. An ECG sensor may be configured to detect electrical activity of a subject's heart. A temperature sensor may be configured to detect temperatures associated with the subject, such as skin temperature and/or core body temperature. A respiratory sensor may be configured to detect respiration of the subject. An accelerometer may be configured to detect movement of at least a portion of the subject. A microphone may be configured to detect sound. A gyrometer sensor may be used to measure angular velocity of at least a portion of the subject, such as the torso of subject. An oxygen saturation sensor may be used to record blood oxygenation of the subject. An EDA sensor may be used to measure EDA of the skin of the subject. A volatile organic compound (VOC) detector may be used to detect various organic molecules that may be coming off of the subject or that may be in the subject's sweat. An optical sensor may be used to monitor or detect changes in color, such as changes in skin coloration of the subject. A spectrometer may measure electromagnetic (EM) radiation and may be configured to detect variations in reflected EM radiation. For example, such a sensor may detect changes in color in a molecule exposed to multi-spectral light (e.g., white light), and/or may detect other changes in reflected EM radiation outside of the visible spectrum (e.g., interaction with ultra-violet rays, etc.).

Referring to FIG. 5D, the torso sensor device 500 includes one or more optical emitters 520 and optical detectors 522 which together are part of a corresponding one of the optical sensors 504, 506. The optical emitters 520 are driven by drivers 523 to emit light 524 which is at least partially transmitted through or reflected by a patient's tissue before reaching the optical detector 522. The optical detector 522 generates electrical signals representing received light (e.g., red and/or IR light) which are provided to a data acquisition block 526. The data acquisition block 526 applies gain control 528 and offset cancellation 530 to the electrical signals received from the optical detector 522. Output of the data acquisition block 526 is provided to signal processing block 532 which includes DC calculation 534 to calculate a DC component of the electrical signals, AC calculation 536 to calculate an AC component of the electrical signals, ratio calculation 538 which calculates various ratios as described elsewhere herein, and oxygen saturation calculation 540 which calculates an oxygen saturation measurement from one or more of the ratios. Some or all of the data acquisition block 526 and/or signal processing block 532 may be included in or correspond to the processor 508 of FIG. 5C. Oxygen saturation measurements, ratios, signals and/or data received at and/or generated by the signal processing block 532 may be output to a configuration and data storage block 542 for storage and/or to an encryption block 544 for encryption and subsequent transportation via transport block 546 to a server 548. The configuration and data storage block 542 may alternatively or additionally store configuration data for, e.g., the signal processing block 532. The encryption block 544 may encrypt outbound communications from the torso sensor device 500 to the server 548 and/or may decrypt inbound communications from the server 548 to the torso sensor device 500. The transport block 546 may encapsulate encrypted data from the encryption block 544 for transport to the server 548 and/or may extract encrypted data received from the server 548 for decryption by the encryption block 544. Portions or all of the encryption block 544 and/or the transport block 546 may be included in or correspond to the communication interface 512 of FIG. 5C.

The server 548 may include a signal processing block 550 to process data from the torso sensor device 500, a trending block 552 to track or identify trends in the data from the torso sensor device 500, a rendering block or graphical user interface (GUI) 554 to render photoplethysmographic traces and/or other data (e.g., oxygen saturation measurements) based on the data from the torso sensor device 500, a statistics block 556 to generate statistics based on the data from the torso sensor device 500, and an alerts/alarms/events block 558 to generate alerts, alarms, and/or events based on the data. Some or all of the server 548 may be included in or correspond to the server 108 of FIG. 1.

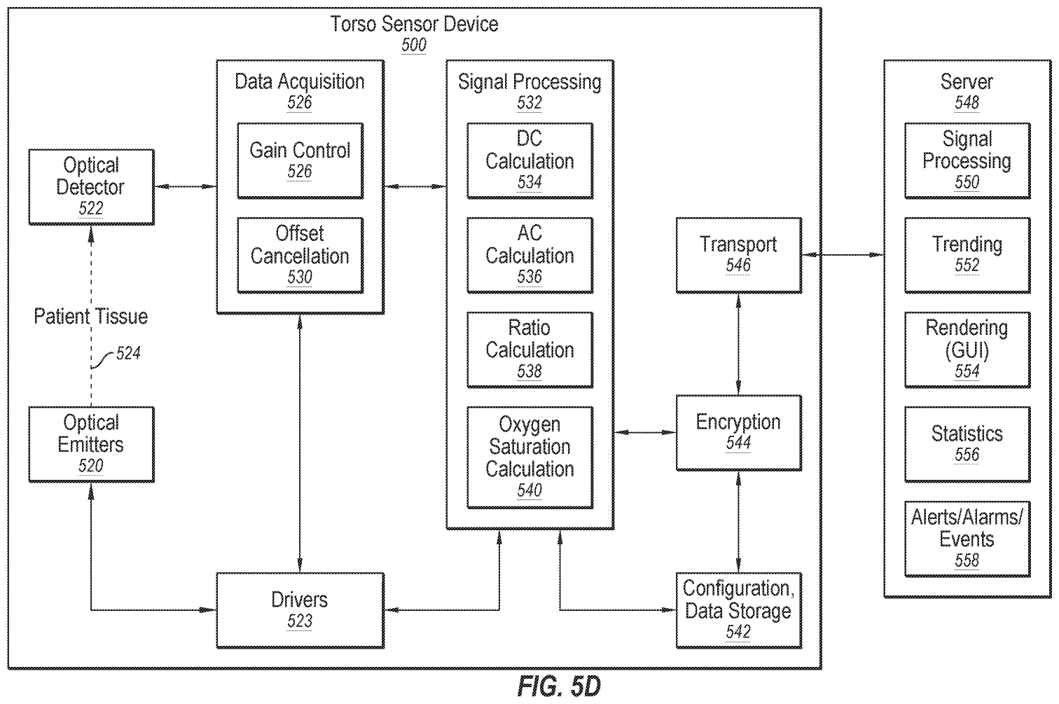

FIG. 6 illustrates another example torso sensor device 600 that may be implemented in the environment 100 of FIG. 1, arranged in accordance with at least one embodiment described herein. The torso sensor device 600 may include, be included in, or correspond to the torso sensor device 102 of FIG. 1 and/or other torso sensor devices described herein. FIG. 6 is a top view of the torso sensor device 600, the torso sensor device 600 including a housing 602 with a first surface (not visible in FIG. 6) configured to be positioned against a subject's torso in use and an opposing second surface 604. The torso sensor device 600 also includes two (or more) optical sensors spaced apart from each other and positioned at the first surface. Approximate footprints or outlines of the optical sensors as projected from the first surface to the second surface 604 are denoted at 606 and 608. As such, the optical sensors of the torso sensor device 600 are hereinafter referred to as the “torso sensor 606” and the “torso sensor 608”. Each of the optical sensors 606, 608 has a corresponding sensor axis 610, 612.

The torso sensor device 600 further includes a first set of one or more alignment guides 614A, 614B (hereinafter collectively “alignment guides 614”) and a second set of one or more alignment guides 616A, 616B (hereinafter collectively “alignment guides 616”) on the second surface 604 of the housing 602. Each of the alignment guides 614, 616 may generally include a protrusion or ridge extending from the housing 602, an indentation in the housing 602, a depression in the housing 602, a sticker applied to the housing 602, a mark printed on the housing 602, or other indicator of the location and/or alignment of the corresponding optical axis 610, 612. For example, each of the alignment guides 614, 616 is an elongate protrusion in the example of FIG. 6.

Further, each set of alignment guides 614, 616 and/or each of the alignment guides 614, 616 individually may be aligned to the corresponding optical axis 610, 612. For example, the alignment guides 614 as a set define a reference line from one of the alignment guides 614 to the other that is parallel to the optical axis 610. In addition, each of the alignment guides 614 is elongate and individually defines a reference line along its length that is parallel to the optical axis 610. Similarly, the alignment guides 616 as a set define a reference line from one of the alignment guides 616 to the other that is parallel to the optical axis 612. In addition, each of the alignment guides 616 is elongate and individually defines a reference line along its length that is parallel to the optical axis 612.

Accordingly, the alignment guides 614 are configured to visually identify the location and/or alignment of the sensor axis 610 relative to the second surface 604 even though the optical sensor 606 may not be visible when the torso sensor device 600 is arranged relative to a viewer with the first surface at which the optical sensor 606 is located behind the second surface 604. Similarly, the alignment guides 616 are configured to visually identify the location and/or alignment of the sensor axis 612 relative to the second surface 604 even though the optical sensor 608 may not be visible when the torso sensor device 600 is arranged relative to a viewer with the first surface at which the optical sensor 608 is located behind the second surface 604. Thus, when the torso sensor device 600 is being coupled to a subject, the subject or other individual may locate one or two ribs of the subject and/or a corresponding ICS by palpation, visual examination, imaging (e.g., X-ray), or in another suitable manner. The subject or other individual may then position the torso sensor device 600 with the first surface against the subject's torso (such that the optical sensors 606, 608 are not visible) and the alignment guides 614 and/or the alignment guides 616 positioned over the ICS such that the optical sensor 606 and/or the optical sensor 608 is positioned over the ICS. Alternatively or additionally, the subject or other individual may position the torso sensor device 600 with the alignment guides 614 and/or the alignment guides 616 aligned parallel to the ICS or one or both of the ribs that are adjacent to the ICS such that the sensor axis 610 and/or the sensor axis 612 is aligned parallel to the ICS or one or both of the ribs that are adjacent to the ICS.

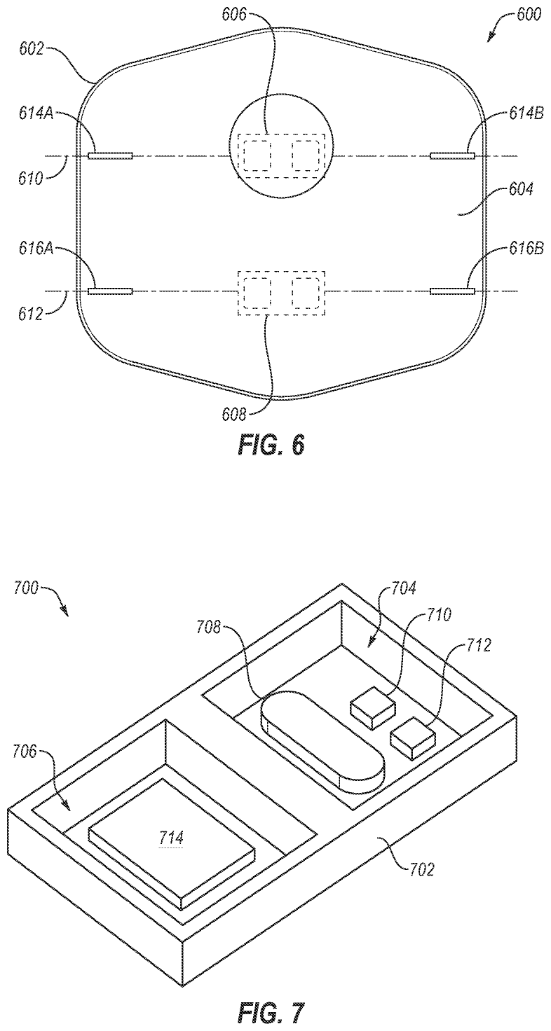

FIG. 7 is a top front perspective view of an example optical sensor 700 that may be implemented in any of the torso sensor devices herein, arranged in accordance with at least one embodiment described herein. The optical sensor 700 may include, be included in, or correspond to any of the optical sensors described herein.

As illustrated, the optical sensor 700 includes a housing 702 having a first cavity 704 for one or more optical emitters and a second cavity 706 for one or more optical detectors. In particular, the optical sensor 700 includes three optical emitters 708, 710, 712 disposed in the first cavity 704 and an optical detector 714 disposed in the second cavity 706. The optical emitter 708 may include an LED, a non-monochromatic light source, a polychromatic light source, a broadband light source, a white light LED, and/or other optical emitter. The optical emitter 708 may be employed to obtain oxygen saturation measurements during motion events and/or in other circumstances, as described in U.S. Pat. No. 10,485,463. The optical emitter 710 may include a monochromatic light source, such as an IR LED, or other optical emitter and is hereinafter referred to as the IR LED 710. The optical emitter 712 may include a monochromatic light source, such as a red LED, or other optical emitter and is hereinafter referred to as the red LED 712. The optical detector 714 may include a dual photodiode, a dual junction wavelength detector, an epitaxial spectral sensor, or other suitable optical detector.

In operation, a portion of the subject that has some arterial blood flow (e.g., an ICS of the subject) is alternately illuminated by the IR LED 710 and the red LED 712. Some of the light that reaches the subject's arterial blood is absorbed by hemoglobin and some of the light is reflected back toward the optical sensor 700 where it may be detected by the optical detector 714. The amount of light that is absorbed depends on, among other things, the wavelength of the illuminating light and the concentrations of hemoglobin carrying oxygen, also referred to as oxyhemoglobin, and hemoglobin without oxygen, also referred to as deoxyhemoglobin. For example, oxyhemoglobin absorbs more IR light than red light and deoxyhemoglobin absorbs more red light than IR light. For each of the IR light and the red light, the optical detector 714 separately detects the amount of non-absorbed and reflected light and generates an IR reflectance signal and a red reflectance signal. In some embodiments, a processor (such as processor 508) included in or coupled to the optical sensor 700 may invert the IR and red reflectance signals output by the optical detector 714 to generate an IR absorption signal or a red absorption signal. Oxygen saturation measurements may be generated from the reflectance signals and/or the absorption signals.

Each of the IR reflectance signal, the IR absorption signal, the red reflectance signal, and the red absorption signal includes a DC component representing reflectance (in the case of reflectance signals) or absorption (in the case of absorption signals) of surrounding tissue, venous blood, and/or other non-pulsing light absorbing elements at the measurement site and an AC component representing reflectance or absorption of pulsatile arterial blood at the measurement site. The optical sensor 700 and/or a processor coupled to the optical sensor 700 may generate a pulsatile signal from the two AC components or from both the two AC components and the two DC components, e.g., by calculating a reflectance or absorption ratio based on and/or using the AC components (and optionally the DC components), converting the reflectance or absorption ratio over time to oxygen saturation over time (e.g., using a table with empirical formulas), and/or by otherwise processing or using the AC components and/or the DC components. The pulsatile signal is or represents oxygen saturation of the subject as a function of time and is generally sinusoidal.

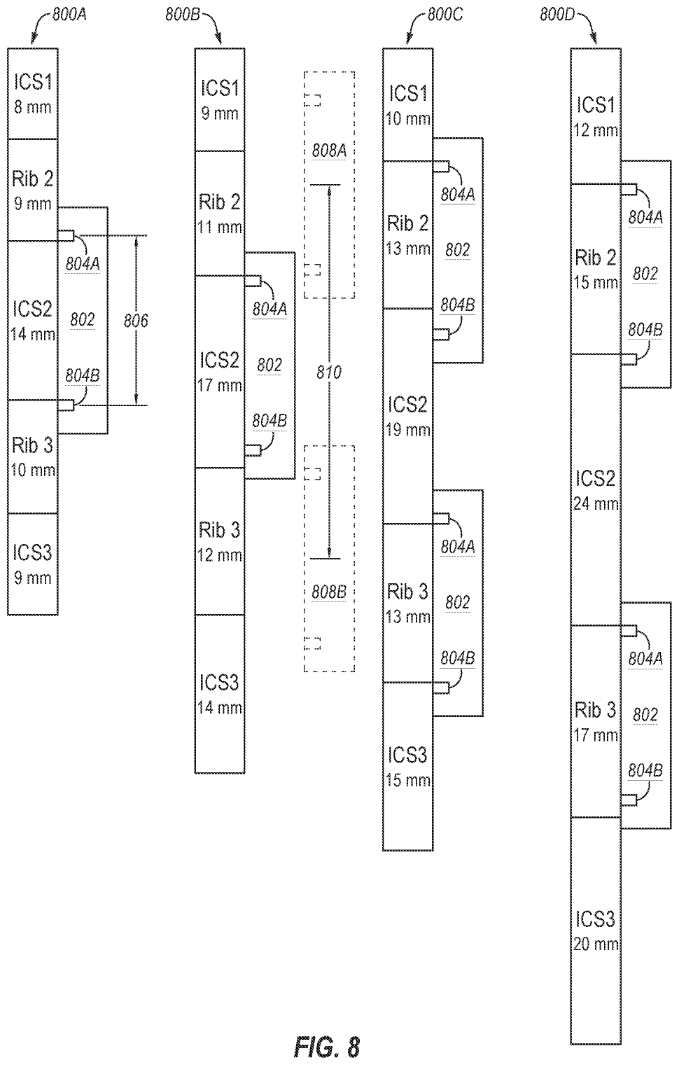

FIG. 8 illustrates various rib models 800A-800D (hereinafter collectively “rib models 800”) and an example configuration of a torso sensor device 802, arranged in accordance with at least one embodiment described herein. In FIG. 8, the rib models 800 and torso sensor devices 802 are to scale. Each torso sensor device 802 illustrated in FIG. 8 may include, be included in, or correspond to any of the torso sensor devices described herein. Each torso sensor device 802 includes first and second optical sensors 804A, 804B (hereinafter collectively “optical sensors 804”).

Each of the rib models 800 may be a rib model for a different population of human adults, such as small females, typical females, typical males, and large males. Alternatively or additionally, each of the rib models 800 includes the first ICS (“ICS1” in FIG. 8), the second rib (“Rib 2” in FIG. 8), the second ICS (“ICS2” in FIG. 8), the third rib (“Rib 3” in FIG. 8), and the third ICS (“IC S3” in FIG. 8) on the anatomical left side. Within a given rib model, a measurement beneath a corresponding label indicates a width (e.g., vertical measurement or measurement generally parallel to a subject's spine) of the labeled element for the given rib model. For example, the measurement of “8 mm” beneath the label “ICS1” in the rib model 800A indicates that the first ICS in the rib model 800A has a width of 8 mm. Each of the rib models 800 may be derived empirically for a given population, from known or inferred relationships between populations, or in some other manner.

The rib model 800B represents some rib and ICS measurements of a typical female. The rib model 800B may be derived empirically, e.g., by measuring the width (e.g., in the vertical direction or generally parallel to the spine) of, among potentially others, the first, second, and third ICSs and the second and third ribs across a population of female subjects and then taking an average, mean, median, or the like for each ICS measurement and each rib measurement across the population. For example, the average, mean, median, or the like of the first ICS measurement across the population of female subjects may be 9 mm, as indicated by the measurement of “9 mm” beneath the label “ICS1” in the rib model 800B. Alternatively or additionally, the rib model 800B may be derived in some other manner.

The rib model 800A represents some rib and ICS measurements of a small female. The rib model 800A may include the smallest measurement(s) from the population of females used to generate the rib model 800B, an average, mean, median of the smallest 10% (or other percentage) of the measurements from the population of females used to generate the rib model 800B, or some other set of measurements or derived values.

The rib model 800C represents some rib and ICS measurements of a typical male. The rib model 800C may be derived empirically, e.g., by measuring the width (e.g., in the vertical direction or generally parallel to the spine) of, among potentially others, the first, second, and third ICSs and the second and third ribs across a population of male subjects and then taking an average, mean, median, or the like for each ICS measurement and each rib measurement across the population. For example, the average, mean, median, or the like of the first ICS measurement across the population of male subjects may be 10 mm, as indicated by the measurement of “10 mm” beneath the label “ICS1” in the rib model 800C. Alternatively or additionally, the rib model 800C may be derived in some other manner.

The rib model 800D represents some rib and ICS measurements of a large male. The rib model 800D may include the largest measurement(s) from the population of males used to generate the rib model 800C, an average, mean, median of the largest 10% (or other percentage) of the measurements from the population of males used to generate the rib model 800C, or some other set of measurements or derived values.

The optical sensors 804 of the torso sensor device 802 have a center to center spacing 806 (hereinafter “spacing 806”). The spacing 806 is only indicated for the torso sensor device 802 next to the rib model 800A for simplicity but the other torso sensor devices 802 depicted in FIG. 8 have the same spacing 806. In an example embodiment, the spacing 806 may be about 50% of the rib 2 center to rib 3 center spacing, which would be approximately 12 mm for the rib model 800A, 14 mm for the rib model 800B, 16 mm for the rib model 800C, or 20 mm for the rib model 800D. Alternatively or additionally, the spacing 806 of the optical sensors 804 in the torso sensor device 802 may be in a range from 5 mm to 150 mm, or in a range from 10 mm to 20 mm, or in a range from 14 mm to 17 mm, or in another range.

In the illustrated example, the spacing 806 of the optical sensors 804 is 15 mm. In this example, and assuming optical axes of the optical sensors 804 are aligned parallel to the ribs and/or ICSs, or in and out of the page in FIG. 8, placement of the torso sensor device 802 generally in the vicinity of ICS 2 in the rib models 800B, 800C results in at least one of the optical sensors 804 being positioned over an ICS (e.g., ICS1 and/or ICS2; or ICS2 and/or ICS3). As used herein, the torso sensor device 802 being “in the vicinity of IC S2” means the torso sensor device 802 is located anywhere in a range that begins with the point at which the optical sensor 804A is directly over the second rib while in the vertical direction in FIG. 8 the optical sensor 804B is adjacent to ICS2 and extends to the point at which the optical sensor 804A is directly over the third rib while in the vertical direction in FIG. 8 the optical sensor 804A is adjacent to ICS2. A first profile 808A of the torso sensor device 802 shows the placement of the torso sensor device 802 relative to the rib model 800B at one end of the range where the chest sensor device 802 is considered in the vicinity of ICS2; this end of the range is referred to as the upper end of the range. A second profile 808B of the torso sensor device 802 shows the placement of the torso sensor device 802 relative to the rib model 800B at the other end of the range where the torso sensor device 802 is considered in the vicinity of ICS2; this end of the range is referred to as the lower end of the range. A reference line 810 shows the complete placement range of a center of the torso sensor device 802 relative to the rib model 800B when the torso sensor device 802 is considered to be in the vicinity of ICS2. The foregoing range of relative placements defined for “in the vicinity of ICS2” can be adapted to define ranges for “in the vicinity of the second rib”, “in the vicinity of ICS3”, and so on.

The spacing 806 of the optical sensors 806 may be selected to minimize or at least reduce a likelihood compared to a torso sensor device having a single optical sensor of both of the optical sensors 806 being positioned simultaneously over a rib when the torso sensor device is coupled to the subject in the vicinity of ICS2. Alternatively or additionally, The spacing 806 of the optical sensors 806 may be selected to minimize or reduce a likelihood compared to a torso sensor device having a single optical sensor of both of the optical sensors 806 being positioned simultaneously over a rib when the torso sensor device is coupled to the subject in the vicinity of other ICS s of the subject.

In the rib models 800B, 800C, the spacing 806 (15 mm in this example) is less than the ICS2 measurement (of 17 mm or 19 mm) so up to both of the optical sensors 804 may be positioned over ICS2. In addition, the spacing 806 (15 mm in this example) is greater than the measurement of the second rib (11 mm or 13 mm) and of the third rib (12 mm or 13 mm) so both optical sensors 804 will never be simultaneously occluded by the second or third ribs alone. In particular, if the torso sensor device 802 is shifted from the positions shown in FIG. 8 upward relative to the ICS2 of the rib models 800B, 800C anywhere from 0 mm to the upper end of the range of placements for which the torso sensor device 802 is considered to be in the vicinity of ICS2, at least one of the optical sensors 804 will be positioned over ICS1 and/or ICS2 since the spacing 806 is greater than the width of the second rib (e.g., 11 mm or 13 mm) in the models 800B, 800C. Similarly, if the torso sensor device 802 is shifted downward from the positions shown in FIG. 8 relative to the ICS2 of the rib models 800B, 800C anywhere from 0 mm to the lower end of the range of placements for which the torso sensor device 802 is considered to be in the vicinity of ICS2, at least one of the optical sensors 804 will be positioned over ICS2 and/or ICS3 since the spacing 806 is greater than the width of the third rib (e.g., 12 mm or 13 mm) in the models 800B, 800C. Thus, all placements of the torso sensor device 802 in the vicinity of ICS2 result in at least one of the optical sensors 804 being positioned over ICS1, ICS2, and/or ICS3.

In addition, most but not all placements of the torso sensor device 802 in the vicinity of ICS2 in the rib models 800A, 800D result in at least one of the optical sensors 804 being positioned over an ICS (e.g., ICS1 and/or ICS2) provided optical axes of the optical sensors 804 are aligned substantially parallel to the ribs and/or ICSs. In the model 800A, for example, the spacing 806 (15 mm) is slightly greater than the ICS2 measurement (14 mm), such that it is possible for the optical sensor 804A to be positioned over the second rib while the optical sensor 804B is positioned over the third rib—this is only one placement in the range of placements in which the torso sensor device 802 is considered to be in the vicinity of ICS2. Every other placement of the torso sensor device 802 in the vicinity of ICS2 where the torso sensor device 802 is shifted from the position shown in FIG. 8 upward or downward relative to the rib model 800A by at least 1 mm results in at least one of the optical sensors 804 being directly over ICS1, ICS2, and/or ICS3.

For example, shifting the torso sensor device 802 from the position shown in FIG. 8 upward relative to the rib model 800A anywhere from 1 mm to 8 mm results in the optical sensor 804B being directly over ICS2 and the optical sensor 804A being directly over the second rib. Shifting the torso sensor device 802 from the position shown in FIG. 8 upward relative to the rib model 800A anywhere from 9 mm to 14 mm results in the optical sensor 804B being directly over ICS2 and the optical sensor 804A being directly over ICS1. Shifting the torso sensor device 802 from the position shown in FIG. 8 upward relative to the rib model 800A by 15 mm (which is the upper end of the range in which the torso sensor device 802 is considered to be in the vicinity of ICS2) results in the optical sensor 804B being directly over the second rib and the optical sensor 804A being directly over ICS1. Shifting the torso sensor device 802 from the position shown in FIG. 8 downward relative to the rib model 800A anywhere from 1 mm to 9 mm results in the optical sensor 804A being directly over ICS2 and the optical sensor 804B being directly over the third rib. Shifting the torso sensor device 802 from the position shown in FIG. 8 downward relative to the rib model 800A anywhere from 10 mm to 14 mm results in the optical sensor 804A being directly over ICS2 and the optical sensor 804B being directly over ICS3. Shifting the torso sensor device 802 from the position shown in FIG. 8 downward relative to the rib model 800A by 15 mm (which is the lower end of the range in which the torso sensor device 802 is considered to be in the vicinity of ICS2) results in the optical sensor 804A being directly over the third rib and the optical sensor 804A being directly over ICS3. Thus, it is only in the middle of the range of the torso sensor device 802 being in the vicinity of ICS2 that both optical sensors 804 are directly over a rib. For every other placement of the torso sensor device 802 in the vicinity of ICS2 where the torso sensor device 802 is shifted from the position shown in FIG. 8 upward or downward relative to the rib model 800A by at least 1 mm, at least one of the optical sensors 804 is directly over ICS1, ICS2, and/or ICS3.

The situation is analogous for the rib model 800D. In the discussion that follows, any reference to a torso sensor device 802 refers specifically to the upper of the two torso sensor devices 802 shown adjacent to the rib model 800D unless explicitly stated otherwise. In the model 800D, the spacing 806 (15 mm) is less than the width of the third rib (17 mm) and equal to the width of the second rib (15 mm), such that it is possible for each of the optical sensors 804 to be simultaneously positioned directly over the third rib or for half of each of the optical sensors 804 to be simultaneously positioned directly over the second rib while the other half of each of the optical sensors is positioned directly over ICS1 or ICS2. Every other placement of the torso sensor device 802 in the vicinity of ICS2 where the torso sensor device 802 is shifted from the position shown in FIG. 8 upward relative to the rib model 800D by 1 mm (the upper end of the range) or downward relative to the rib model 800D by 0 mm to 38 mm results in at least one of the optical sensors 804 being directly over ICS1, ICS2, and/or ICS3.

For example, shifting the torso sensor device 802 from the position shown in FIG. 8 upward relative to the rib model 800D by 1 mm, which is the upper end of the range in which the torso sensor device 802 is considered to be in the vicinity of ICS2, results in the optical sensor 804A being directly over ICS 1 and the optical sensor 804B being directly over the second rib. Any placement of the torso sensor device 802 starting with the position shown in FIG. 8 to a downward shift of the torso sensor device 802 relative to the rib model 800D of up to 14 mm results in the optical sensor 804A being directly over the second rib and the optical sensor 804B being directly over ICS2. Shifting the torso sensor device 802 from the position shown in FIG. 8 downward relative to the rib model 800D anywhere from 14 mm to 23 mm results in both optical sensors 804 being directly over ICS2. Shifting the torso sensor device 802 from the position shown in FIG. 8 downward relative to the rib model 800D anywhere from 24 mm to 38 mm results in the optical sensor 804A being directly over ICS2 and the optical sensor 804B being directly over the third rib. Shifting the torso sensor device 802 from the position shown in FIG. 8 downward relative to the rib model 800D by 39 mm to the location of the lower torso sensor device 802, which is the lower end of the range in which the torso sensor device 802 is considered to be in the vicinity of ICS2, results in both optical sensors 804 being directly over the third rib. Thus, it is only at the lower ends of the range of the torso sensor device 802 being in the vicinity of ICS2 that both optical sensors 804 are directly over a rib and only at 0.5 mm from the upper end of the range of the torso sensor device 802 being in the vicinity of ICS2 that half of each of the optical sensors 804 is directly over a rib. For every other placement of the torso sensor device 802 in the vicinity of ICS2 where the torso sensor device 802 is shifted from the position shown in FIG. 8 upward relative to the rib model by 1 mm (the upper end of the range) or downward relative to the rib model 800D by 0 mm to 38 mm, at least one of the optical sensors 804 is directly over ICS1, ICS2, and/or ICS3.

It can be seen from FIG. 8 that the spacing 806 of the optical sensors 804, when selected appropriately, may be configured to prevent the optical sensors 804 from being simultaneously occluded by the second rib or the third rib (i.e., simultaneous placement of both optical sensors 804 directly over the second rib or of both optical sensors 804 directly over the third rib) at least in the case of the rib models 800A-800C when optical axes of the optical sensors 804 are aligned parallel to a subject's second rib, ICS2, or the like. It can be further seen from FIG. that the spacing 806, when selected appropriately, may be further configured to prevent simultaneous occlusion of the optical sensor 804A by the second rib and the optical sensor 804B by the third rib (i.e., simultaneous placement of the optical sensor 804A directly over the second rib and the optical sensor 804B directly over the third rib) at least in the case of the rib models 800B-800D when optical axes of the optical sensors 804 are aligned parallel to a subject's second rib, ICS2, or the like.

Alternatively or additionally, multiple torso sensor devices 802 with different spacings 806 may be provided or available for use on subjects. For example, torso sensor devices 802 with smaller spacing 806 may be used on smaller individuals with smaller ribs and narrower ICS while torso sensor devices 802 with larger spacing 806 may be used on larger individuals with larger ribs and wider ICS. As a particular example, torso sensor devices 802 with spacing 806 of 13 mm (or some other spacing less than 15 mm) may be available for use on smaller individuals with smaller ribs and narrower ICSs while torso sensor devices 802 with spacing 806 of 18 mm (or some other spacing more than 15 mm) may be available for use on larger individuals with larger ribs and wider ICSs. In an example embodiment, two or more “sizes” may be available, such as a small size torso sensor device 802 with a spacing 806 of 13 mm, a medium or regular size torso sensor device 802 with a spacing 806 of 15 mm, and a larger size torso sensor device 802 with a spacing 806 of 18 mm. In this and other embodiments, a healthcare provider may select a desired size torso sensor device 802 based on a size of a subject.

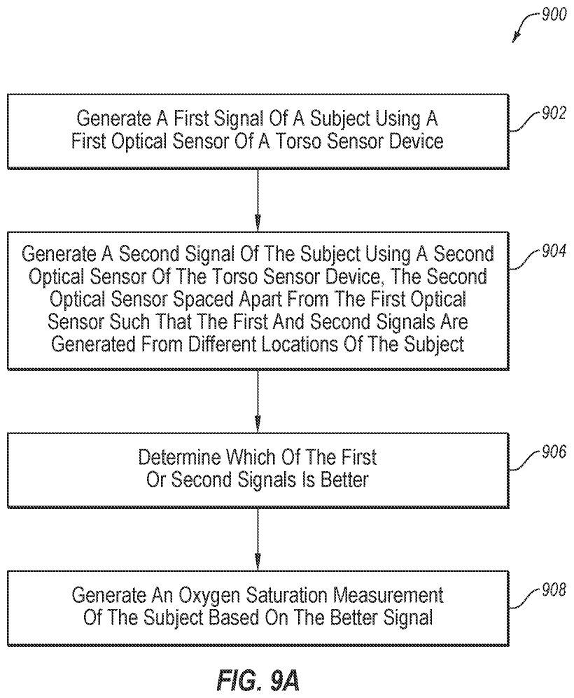

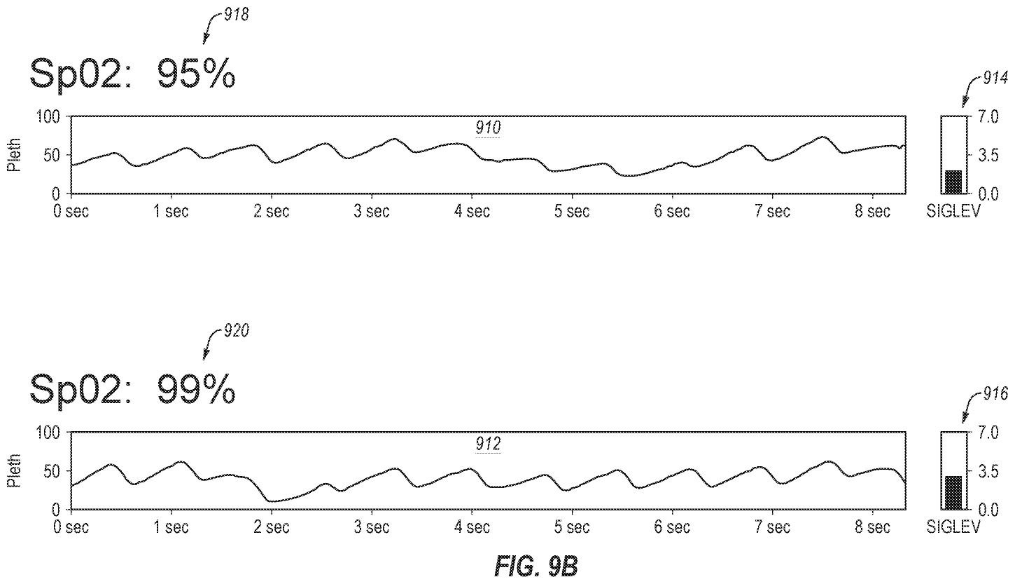

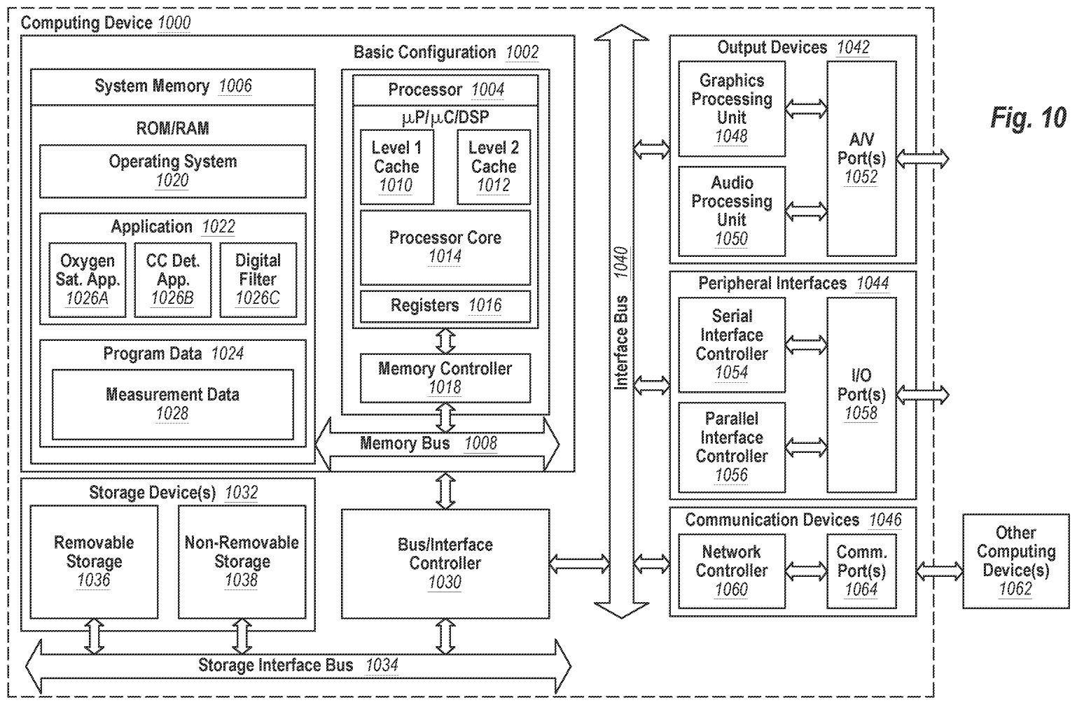

FIG. 9A is a flowchart of a method 900 to measure oxygen saturation, arranged in accordance with at least one embodiment described herein. The method 900 may be programmably performed or controlled by one or more processors, in, e.g., one or more torso sensor devices, such any of the torso sensor devices disclosed herein. In an example implementation, the method 900 may be performed and/or controlled in whole or in part by a computing device 1000 depicted in FIG. 10 that may include, be included in, or correspond to any of the torso sensor devices herein. The method 900 may include one or more of blocks 902, 904, 906, and/or 908.

At block 902, the method 900 may including generating a first signal of a subject using a first optical sensor of a torso sensor device. For example, block 902 may include generating the first signal of the subject using the optical sensor 302, 504, 606, 804A of the torso sensor device 300, 500, 600, 802. The first signal may include an IR or red reflectance or absorption signal or a corresponding pulsatile signal and/or may be generated as described with respect to, e.g., FIG. 7. Block 902 may be followed by block 904.

At block 904, the method 900 may include generating a second signal of the subject using a second optical sensor of the torso sensor device, the second optical sensor spaced apart from the first optical sensor such that the first and second signals are generated from different locations of the subject. For example, block 904 may include generating the second signal of the subject using the optical sensor 304, 506, 608, 804B of the torso sensor device 300, 500, 600, 802. The second signal may include an IR or red reflectance or absorption signal or a corresponding pulsatile signal and/or may be generated as described with respect to, e.g., FIG. 7. Block 904 may be followed by block 906.

At block 906, the method 900 may include determining which of the first or second signals is better. For example, block 906 may generally include determining which of the first and second signals has a greater AC swing or peak to peak undulation. Block 906 may be followed by block 908.

At block 908, the method 900 may include generating an oxygen saturation measurement of the subject based on the better signal. Where the better signal includes an IR reflectance signal, an IR absorption signal, a red reflectance signal, or a red absorption signal, generating the oxygen saturation measurement at block 908 may include, for example, removing a DC component of the signal, generating a pulsatile signal, calculating ratios of AC to DC components of a signal (e.g., normalization of the AC component by the DC component), calculating ratios of a normalized AC component of one signal to a normalized AC component of another signal (ratios of ratios), and/or using the pulsatile signal as the oxygen saturation measurement.

In some embodiments, the better signal is a red absorption signal or an IR absorption signal. If the better signal is the red absorption signal, the IR absorption signal from the same sensor as the sensor that generated the red absorption signal may also be used in the generation of the oxygen saturation measurement. If the better signal is the IR absorption signal, the red absorption signal from the same sensor as the sensor that generated the IR absorption signal may also be used in the generation of the oxygen saturation measurement. In both examples, generating the oxygen saturation measurement based on the better signal may include calculating a ratio R R of the AC component of the red absorption signal to the DC component of the red absorption signal, calculating a ratio R_{I} of the AC component of the IR absorption signal to the DC component of the IR absorption signal, and calculating an absorption ratio R of the ratios R_{R} and R_{I}, i.e., R=R_{R}/R_{I}. The oxygen saturation may then be calculated as a function of the absorption ratio R. A simplified version of the relationship between the absorption ratio R and the oxygen saturation is SpO₂ %=110−25*R. The actual relationship between the oxygen saturation and the absorption ratio R may be more complex in practice.

If the better signal is a reflectance signal, it may be converted to an absorption signal and/or the calculations may be modified to account for the relationship between absorption and reflectance.

One skilled in the art will appreciate that, for this and other processes and methods disclosed herein, the functions performed in the processes and methods may be implemented in differing order. Further, the outlined steps and operations are only provided as examples, and some of the steps and operations may be optional, combined into fewer steps and operations, or expanded into additional steps and operations without detracting from the essence of the disclosed embodiments.

For example, in some embodiments, block 906 may include determining peak-to-trough differences in each of the first and second signals and comparing the peak-to-trough difference of the first signal to the peak-to-trough difference of the second signal to identify which is greater. The signal with the greater peak-to-trough difference may be the better signal. Alternatively or additionally, the method 900 may further include extracting amplitude values of peaks and troughs in the first signal over a first duration of time and in the second signal over a second duration of time. In these and other embodiments, determining the peak-to-trough difference in the first signal may include averaging a series of peak-to-trough differences calculated from the extracted amplitude values of the first duration of time; and determining the peak-to-trough difference in the second signal may include averaging a series of peak-to-trough differences calculated from the extracted amplitude values of the second duration of time.

In an alternative implementation of the method 900, block 906 may instead or additionally include generating a third signal from a combination of the first and second signals generated at blocks 902 and 904. The first and second signals may be summed, averaged, or otherwise combined to generate the third signal. Alternatively or additionally, the method 900 may determine which of the first or second signals is better and weight the “better” signal more or higher than the other signal when generating the third signal. Alternatively or additionally, block 908 may include generating an oxygen saturation measurement based on the third signal.

Example aspects of block 906 will now be described with respect to FIG. 9B, which illustrates example photoplethysmographic traces 910, 912 from two different optical sensors of the same torso sensor device, such as the torso sensor device 300, 500, 600, arranged in accordance with at least one embodiment herein. Each photoplethysmographic trace 910, 912, or “pleth”, graphically represents the normalized AC component of the IR absorption signal as a function of time as generated from a corresponding one of two optical sensors of the torso sensor device. For example, the pleth 910 graphically represents the normalized AC component of the IR absorption signal generated from a first optical sensor of the torso sensor device while the pleth 912 graphically represents the normalized AC component of the IR absorption signal generated from a second optical sensor of the torso sensor device. The first optical sensor in this example was positioned over a rib of a subject while the second optical sensor in this example was positioned over an ICS of the subject. The normalized AC component of the IR absorption signal may be referred to as AC_{I}. Analogously, the normalized AC component of the red absorption signal may be referred to as AC_{R}.

FIG. 9B additionally illustrates a signal level (SIGLEV in FIG. 9B) 914, 916 for each of the optical sensors of the torso sensor device. In this example, the signal level 914, 916 is the scale factor by which the corresponding “pleth” signals 910 and 912, respectively are scaled. That is, SIGLEV indicates the amplitude of the AC component of the IR absorption signal. SIGLEV is indicated on a logarithmic scale and represents the amplitude factor by which the “pleth” signal is scaled in order to normalize the signal for display. The signal level 914, 916 may include or correspond to a peak-to-trough difference, such as an instantaneous peak-to-trough difference, an average peak-to-trough difference, or the like, of each pleth 910, 912 (e.g., of the AC component of the IR absorption signal). Block 906 of the method 900 may include comparing the signal level 914 to the signal level 916 to determine that a signal (e.g., AC_{R}, AC_{I}, R_{R}, R_{I}, and/or R) from the second optical sensor positioned over the subject's ICS is better than the corresponding signal (e.g., AC_{R}, AC_{I}, R_{R}, R_{I}, and/or R) from the first optical sensor positioned over the subject's rib. In particular, in the example of FIG. 9B, the signal level 916 is higher than the signal level 914, indicating that the signal from the second optical sensor positioned over the subject's ICS is better (e.g., has a greater AC swing) than the signal from the first optical sensor positioned over the subject's rib. Alternatively and/or additionally, similar “pleth” signals with corresponding signal levels, ratios, and/or ratio of ratios can be generated for each of two different optical sensors for the red spectrum and/or for the full spectrum and/or for corresponding reflectance signals, and each or any of these signals and/or associated derivations from them can then further be evaluated as to which is the better signal.

FIG. 9B also illustrates an oxygen saturation measurement 918, 920 generated from each pleth 910, 912. Block 908 of the method 900 indicates that an oxygen saturation measurement is generated based on the better signal, such as the oxygen saturation measurement 920 of FIG. 9B being generated based on the signal or signals corresponding to the pleth 912 in this example. However, this does not preclude the possibility of also generating an oxygen saturation measurement based on the other signal, such as the oxygen saturation measurement 918 of FIG. 9B being generated based on the signal or signals corresponding to the pleth 910 in this example. In this and other embodiments, whether a single oxygen saturation measurement 918 or 920 is generated or both oxygen saturation measurements 918 and 920 are generated, only the oxygen saturation measurement 920 generated based on the better signal may be output.

As another example of variations that may be made to the method 900 of FIG. 9A, in some embodiments, block 908 may be performed before block 906. That is, oxygen saturation measurements may be generated from both a first signal and a second signal. And then, subsequently, a determination may be made as to which oxygen saturation measurement is the best measurement. For example, the higher oxygen saturation measurement may be selected as the best measurement. Referring to FIG. 9B, for instance, both oxygen saturation measurements may be generated in block 908 and subsequently at block 906, the oxygen saturation measurement 920 may be selected as the best measurement since it is higher than the oxygen saturation measurement 918.

In some embodiments, the method 900 includes sensing cardiac cycles of the subject. The cardiac cycles may be sensed using a cardiac sensor of the torso sensor device or may be extracted from the first and/or second signal(s). In these and other embodiments, the first and second durations of time may include a predetermined number of sensed cardiac cycles of the subject. Alternatively or additionally, the first and second durations of time may be equal and/or may be time aligned such that the two durations of time start at the same start time and end at the same end time.

In some embodiments, the method 900 further includes attaching the torso sensor device to the subject's torso. Attachment of the torso sensor device to the subject's torso may be performed by the subject, a healthcare provider, or other individual. Attaching the torso sensor to the subject's torso may include locating an ICS of the subject; positioning at least one of the first optical sensor or the second optical sensor over the ICS of the subject; and coupling the torso sensor device to the subject's torso with at least one of the first optical sensor or the second optical sensor positioned over the ICS of the subject. In some embodiments, locating the ICS includes locating the first, second, and/or third ICS of the anatomical left side of the subject's rib cage. Alternatively or additionally, the ICS of the subject may be located by palpation, visual examination, imaging (e.g., X-ray), or in another suitable manner.

In some embodiments, the torso sensor device may include first and second sets of alignment guides such as described with respect to the torso sensor device 600 of FIG. 6. In these and other embodiments, the method 900 may further include positioning the first set of alignment guides and/or the second set of alignment guides over the second ICS and/or the third ICS to position the first optical sensor and/or the second optical sensor over the ICS of the subject. The method 900 may further include aligning the first set of alignment guides and/or the second set of alignment guides parallel to the ICS of the subject's rib cage, such as the first ICS, the second ICS, and/or the third ICS. Alternatively or additionally, the method 900 may further include aligning the first set of alignment guides and/or the second set of alignment guides parallel to the ICS of the subject and/or parallel to an adjacent rib, i.e., a rib adjacent to the ICS.

FIG. 10 is a block diagram illustrating an example computing device 1000, arranged in accordance with at least one embodiment described herein. The computing device 1000 may include, be included in, or otherwise correspond to, e.g., the personal electronic devices 106, the server 108, the torso sensor device 102, 200, 300, 500, 600, 802, or other computing devices. In a basic configuration 1002, the computing device 1000 typically includes one or more processors 1004 and a system memory 1006. A memory bus 1008 may be used to communicate between the processor 1004 and the system memory 1006.

Depending on the desired configuration, the processor 1004 may be of any type including, but not limited to, a microprocessor (RP), a microcontroller (RC), a digital signal processor (DSP), or any combination thereof. The processor 1004 may include one or more levels of caching, such as a level one cache 1010 and a level two cache 1012, a processor core 1014, and registers 1016. The processor core 1014 may include an arithmetic logic unit (ALU), a floating point unit (FPU), a digital signal processing core (DSP Core), or any combination thereof. An example memory controller 1018 may also be used with the processor 1004, or in some implementations the memory controller 1018 may include an internal part of the processor 1004.

Depending on the desired configuration, the system memory 1006 may be of any type including volatile memory (such as RAM), nonvolatile memory (such as ROM, flash memory, etc.), or any combination thereof. The system memory 1006 may include an operating system 1020, one or more applications 1022, and program data 1024. The application 1022 may include an oxygen saturation application 1026 that is arranged to perform or control performance of a method to measure oxygen saturation, such as the method 900 of FIG. 9A, and/or other methods or operations described herein. Alternatively or additionally, the application 1022 may include a cardiac cycle detection application (“CC Det. App.” in FIG. 10) 1026B to detect cardiac cycles in sensor data and/or data derived from the sensor data. Alternatively or additionally, the application 1022 may include or control a digital filter 1026C to filter sensor data, such as SpO₂ signals, ECG signals, or the like. The program data 1024 may include measurement data 1028 that may be generated and/or used by the oxygen saturation application 1026A in the measurement of oxygen saturation of a subject, that may be generated and/or used by the cardiac cycle detection application 1026B in the detection of cardiac cycles of the subject, and/or that may be digitally filtered by the digital filter 1026C. In some embodiments, the application 1022 may be arranged to operate with the program data 1024 on the operating system 1020 such that one or more methods or operations may be provided as described herein, including the method 900 of FIG. 9A.

The computing device 1000 may have additional features or functionality, and additional interfaces to facilitate communications between the basic configuration 1002 and any involved devices and interfaces. For example, a bus/interface controller 1030 may be used to facilitate communications between the basic configuration 1002 and one or more data storage devices 1032 via a storage interface bus 1034. The data storage devices 1032 may be removable storage devices 1036, non-removable storage devices 1038, or a combination thereof. Examples of removable storage and non-removable storage devices include magnetic disk devices such as flexible disk drives and hard-disk drives (HDDs), optical disk drives such as compact disk (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer-readable instructions, data structures, program modules, or other data.

The system memory 1006, the removable storage devices 1036, and the non-removable storage devices 1038 are examples of computer storage media or non-transitory computer-readable media. Computer storage media or non-transitory computer-readable media includes RAM, ROM, EEPROM, flash memory or other memory technology, CD-ROM, digital versatile disks (DVDs) or other optical storage, magnetic cassettes, magnetic tape, magnetic disk storage or other magnetic storage devices, or any other non-transitory medium which may be used to store the desired information and which may be accessed by the computing device 1000. Any such computer storage media or non-transitory computer-readable media may be part of the computing device 1000.

The computing device 1000 may also include an interface bus 1040 to facilitate communication from various interface devices (e.g., output devices 1042, peripheral interfaces 1044, and communication devices 1046) to the basic configuration 1002 via the bus/interface controller 1030. The output devices 1042 include a graphics processing unit 1048 and an audio processing unit 1050, which may be configured to communicate to various external devices such as a display or speakers via one or more A/V ports 1052. Diagrams, flowcharts, organizational charts, connectors, and/or other graphical objects generated by the diagram application 1026 may be output through the graphics processing unit 1048 to such a display. The peripheral interfaces 1044 include a serial interface controller 1054 or a parallel interface controller 1056, which may be configured to communicate with external devices such as input devices (e.g., keyboard, mouse, pen, voice input device, touch input device, etc.), sensors, or other peripheral devices (e.g., printer, scanner, etc.) via one or more I/O ports 1058. Such input devices may be operated by a user to provide input to the diagram application 1026, which input may be effective to, e.g., generate curved connectors, designate points as designated points of one or more curved connectors, relocate one or more designated points, and/or to accomplish other operations within the diagram application 1026. The communication devices 1046 include a network controller 1060, which may be arranged to facilitate communications with one or more other computing devices 1062 over a network communication link via one or more communication ports 1064.

The network communication link may be one example of a communication media. Communication media may typically be embodied by computer-readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term “computer-readable media” as used herein may include both storage media and communication media.

The computing device 1000 may be implemented as a portion of a small-form factor portable (or mobile) electronic device such as a smartphone, a personal data assistant (PDA) or an application-specific device. The computing device 1000 may also be implemented as a personal computer including tablet computer, laptop computer, and/or non-laptop computer configurations, or a server computer including both rack-mounted server computer and blade server computer configurations.

Embodiments described herein may be implemented using computer-readable media for carrying or having computer-executable instructions or data structures stored thereon. Such computer-readable media may be any available media that may be accessed by a general-purpose or special-purpose computer. By way of example, such computer-readable media may include non-transitory computer-readable storage media including RAM, ROM, EEPROM, CD-ROM or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable media.

Computer-executable instructions may include, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A torso sensor device, comprising:
a housing configured to be coupled to a subject's torso;
a first optical sensor disposed in the housing;
a second optical sensor disposed in the housing spaced apart from the first optical sensor;
a processor electrically coupled to each of the first and second optical sensors and configured to control the first and second optical sensors to generate oxygen saturation measurements of the subject.

2. The torso sensor device of claim 1, wherein the housing includes a first surface configured to be positioned against the subject's torso and an opposing second surface, each of the first and second optical sensors positioned at the first surface of the housing.

3. The torso sensor device of claim 2, wherein the housing further includes:
a first set of one or more alignment guides on the second surface and aligned parallel to a first sensor axis of the first optical sensor; and
a second set of one or more alignment guides on the second surface and aligned parallel to a second sensor axis of the second optical sensor, wherein the sensor axes of the first and second optical sensors are parallel.

4. The torso sensor device of claim 3, wherein:
the first set of one or more alignment guides is configured to visually identify a location, alignment, or both a location and alignment, of the first sensor axis of the first optical sensor;
the second set of one or more alignment guides is configured to visually identify a location, alignment, or both a location and alignment, of the second sensor axis of the second optical sensor; and
the first set of one or more alignment guides and the second set of one or more alignment guides are further configured to aid alignment of at least one of the first sensor axis or the second sensor axis to an intercostal space (ICS) of the subject's rib cage.

5. The torso sensor device of claim 4, wherein the ICS comprises the first ICS, the second ICS, or the third ICS of the subject's rib cage and the first set of one or more alignment guides and the second set of one or more alignment guides are further configured to aid alignment of at least one of the first sensor axis or the second sensor axis to an adjacent rib of the subject's rib cage.

6. The torso sensor device of claim 1, wherein a center-to-center spacing of the first and second optical sensors is in a range from 5 millimeters to 150 millimeters.

7. The torso sensor device of claim 6, wherein the center-to-center spacing of the first and second optical sensors is in a range from 14 millimeters to 17 millimeters.

8. The torso sensor device of claim 1, wherein a center-to-center spacing of the first and second optical sensors is more than a width of a typical or large male subject's second rib or third rib in the parasternal area of the subject.

9. The torso sensor device of claim 1, wherein a center-to-center spacing of the first and second optical sensors is less than a width of a typical or small female subject's second intercostal space (ICS) in the parasternal area of the subject.

10. The torso sensor device of claim 1, wherein when the torso sensor device is coupled to the subject's torso in a vicinity of the subject's first or second intercostal space (ICS) with sensor axes of the first and second optical sensors aligned substantially parallel to the subject's second rib:
a center-to-center spacing of the first and second optical sensors is configured to prevent the first and second optical sensors from being simultaneously occluded by the subject's second rib; and
the center-to-center spacing of the first and second optical sensors is further configured to prevent the first and second optical sensors from being simultaneously occluded by, respectively, the subject's second and third ribs.

11. The torso sensor device of claim 1, further comprising a non-transitory computer-readable storage medium comprising instructions executable by the processor to perform or control performance of operations comprising:
generating a first signal using the first optical sensor;
generating a second signal using the second optical sensor;
determining which of the first or second signals is better; and
generating an oxygen saturation measurement of the subject based on the better signal.

12. The torso sensor device of claim 11, wherein determining which of the first or second signals is better comprises:
determining a peak-to-trough difference in the first signal;
determining a peak-to-trough difference in the second signal; and
comparing the peak-to-trough difference of the first signal to the peak-to-trough difference of the second signal to identify which is greater, the first or second signal having the greater peak-to-trough difference being identified as the better signal.

13. The torso sensor device of claim 1, further comprising a non-transitory computer-readable storage medium comprising instructions executable by the processor to perform or control performance of operations comprising:
generating a first signal using the first optical sensor;
generating a second signal using the second optical sensor;
generating a third signal from a combination of the first and second signals; and
generating an oxygen saturation measurement of the subject based on the third signal.

14. The torso sensor device of claim 1, wherein each of the first and second optical sensors comprises one or more optical emitters and one or more optical detectors.

15. The torso sensor device of claim 14, wherein the one or more optical emitters of each of the first and second optical sensors comprises at least a red light source and an infrared light source.

16. The torso sensor device of claim 14, wherein the one or more optical emitters of each of the first and second optical sensors further comprises a polymonochromatic light source.

17. The torso sensor device of claim 14, wherein the one or more optical detectors of each of the first and second optical sensors comprises at least a first detector and a second detector, with each of the detectors having different respective spectral response curves.

18. A method to measure oxygen saturation, comprising:
generating a first signal of a subject using a first optical sensor of a torso sensor device;
generating a second signal of the subject using a second optical sensor of the torso sensor device, the second optical sensor spaced apart from the first optical sensor such that the first and second signals are generated from different locations of the subject;
determining which of the first or second signals is better; and
generating an oxygen saturation measurement of the subject based on the better signal.

19. The method of claim 18, wherein determining which of the first or second signals is better comprises:
determining a peak-to-trough difference in the first signal;
determining a peak-to-trough difference in the second signal; and
comparing the peak-to-trough difference of the first signal to the peak-to-trough difference of the second signal to identify which is greater, the first or second signal having the greater peak-to-trough difference being identified as the better signal.

20. The method of claim 19, further comprising:
extracting amplitude values of peaks and troughs in the first signal over a first duration of time, wherein determining the peak-to-trough difference in the first signal comprises averaging a series of peak-to-trough differences calculated from the extracted amplitude values of the first duration of time; and
extracting amplitude values of peaks and troughs in the second signal over a second duration of time, wherein determining the peak-to-trough difference in the second signal comprises averaging a series of peak-to-trough differences calculated from the extracted amplitude values of the second duration of time.

21. The method of claim 20, further comprising sensing cardiac cycles of the subject, wherein each of the first and second durations of time includes a predetermined number of sensed cardiac cycles of the subject.

22. The method of claim 20, wherein the first and second durations of time are equal durations of time.

23. The method of claim 20, wherein the first and second durations of time partially or fully overlap.

24. The method of claim 18, further comprising attaching the torso sensor device to the subject's torso, including:
locating an intercostal space (ICS) of the subject;
positioning at least one of the first optical sensor or the second optical sensor over the ICS of the subject; and
coupling the torso sensor device to the subject's torso with at least one of the first optical sensor or the second optical sensor positioned over the ICS of the subject.

25. The method of claim 24, wherein:
the torso sensor device includes:
a first surface configured to be positioned against the subject's torso and an opposing second surface, each of the first and second optical sensors positioned at the first surface of the housing;
a first set of one or more alignment guides on the second surface and aligned to a first sensor axis of the first optical sensor; and
a second set of one or more alignment guides on the second surface and aligned to a second sensor axis of the second optical sensor;
the sensor axes of the first and second optical sensors are parallel;
the first set of one or more alignment guides is configured to visually identify a location, alignment, or both a location and alignment, of the first sensor axis of the first optical sensor;
the second set of one or more alignment guides is configured to visually identify a location, alignment, or both a location and alignment, of the second sensor axis of the second optical sensor;
the method further comprises positioning at least one of the first set of alignment guides or the second set of alignment guides over the ICS to position the at least one of the first optical sensor or the second optical sensor over the ICS of the subject.

26. The method of claim 25, further comprising aligning at least one of the first set of alignment guides or the second set of alignment guides parallel to the ICS, or an adjacent rib of the subject to align at least one of the first sensor axis or the second sensor axis parallel to the ICS or the adjacent rib.

27. A method to measure oxygen saturation, comprising:
generating a first signal of a subject using a first optical sensor of a torso sensor device;
generating a second signal of the subject using a second optical sensor of the torso sensor device, the second optical sensor spaced apart from the first optical sensor such that the first and second signals are generated from different locations of the subject;
generating a first oxygen saturation measurement of the subject based on the first signal;
generating a second oxygen saturation measurement of the subject based on the second signal;
determining which of the first or second oxygen saturation measurements is better; and
outputting the better oxygen saturation measurement.

28. The method of claim 27, wherein determining which of the first or second oxygen saturation measurements is better comprises:
determining which of the first oxygen saturation measurement or the second oxygen saturation measurement is greater; and
selecting as the better oxygen saturation measurement the first oxygen saturation measurement or the second oxygen saturation measurement that is greater of the two.

29. The method of claim 27, further comprising attaching the torso sensor device to the subject's torso, including:
locating an intercostal space (ICS) of the subject;
positioning at least one of the first optical sensor or the second optical sensor over the ICS of the subject; and
coupling the torso sensor device to the subject's torso with at least one of the first optical sensor or the second optical sensor positioned over the ICS of the subject.

30. The method of claim 29, wherein:
the torso sensor device includes:
a first surface configured to be positioned against the subject's torso and an opposing second surface, each of the first and second optical sensors positioned at the first surface of the housing;
a first set of one or more alignment guides on the second surface and aligned to a first sensor axis of the first optical sensor; and
a second set of one or more alignment guides on the second surface and aligned to a second sensor axis of the second optical sensor;
the sensor axes of the first and second optical sensors are parallel;
the first set of one or more alignment guides is configured to visually identify a location, alignment, or both a location and alignment, of the first sensor axis of the first optical sensor;
the second set of one or more alignment guides is configured to visually identify a location, alignment, or both a location and alignment, of the second sensor axis of the second optical sensor; and
the method further comprises positioning at least one of the first set of alignment guides or the second set of alignment guides over the ICS to position the at least one of the first optical sensor or the second optical sensor over the ICS of the subject.

31. The method of claim 30, further comprising aligning at least one of the first set of alignment guides or the second set of alignment guides parallel to the ICS, or an adjacent rib of the subject to align at least one of the first sensor axis or the second sensor axis parallel to the ICS or the adjacent rib.
